(19) United States
(12) Patent Application Publication
(10) Pub. No.: US 2026/0234547 A1
(43) Pub. Date: Aug. 13, 2026

(54) LACTICASEIBACILLUS RHAMNOSUS STRAIN AND IMMUNITY ENHANCEMENT USES THEREOF

(71) Applicant: KoBioLabs, Inc., Gwanak-gu, Seoul (KR)

(72) Inventors: Hyun Ju YOU, Incheon (KR); Bo-Ram CHO, Seoul (KR); Kiuk LEE, Seoul (KR); Chanseop PARK, Seoul (KR); Seung-Yeon SHIN, Seoul (KR)

(21) Appl. No.: 19/156,131

(22) PCT Filed: Feb. 14, 2024
(86) PCT No.: PCT/KR2024/002112

§ 371 Date: Aug. 13, 2025

(30) Foreign Application Priority Data
Feb. 15, 2023 (KR) 10-2023-0020134

(51) Int. Cl. C12N 1/205; A61K 35/00; A61P 37/04; C12R 1/225

(52) U.S. Cl. CPC C12N 1/205; A61P 37/04; A61K 2035/115; C12R 2001/225

(57) ABSTRACT

The present application relates to a Lacticaseibacillus rhamnosus strain and immune enhancement uses thereof.

### TECHNICAL FIELD

The present application relates to a Lacticaseibacillus rhamnosus strain and immunity enhancement uses thereof.

### BACKGROUND ART

Immunity refers to a defensive mechanism in which the internal environment of a living organism recognizes various substances that have invaded from the outside as foreign materials and eliminates or metabolizes them. However, immune function disorder may occur due to various causes, and various immune-enhancing agents are required to prevent or treat them.

The gut microbiota residing in the human gastrointestinal tract provides various health benefits through close interaction with the human host. Recently, the demand for probiotics has increased significantly worldwide, and there is a need to develop health functional foods and drugs using probiotics.

### DISCLOSURE

### Technical Problem

Under these circumstances, the present inventors have identified that a Lacticaseibacillus rhamnosus KBL352 strain exhibits an immune-enhancing effect by activating immune cells. Accordingly, an embodiment of the present application is directed to providing of a use of the Lacticaseibacillus rhamnosus KBL352 strain for enhancing immunity.

### Technical Solution

An embodiment of the present application relates to a Lacticaseibacillus rhamnosus KBL352 strain deposited under Accession No. KCTC 15267BP, a use of the strain for enhancing immunity, or a use of the strain for preventing or treating immunosuppression. The strain may have a 16S rRNA sequence set forth in SEQ ID NO. 3.

Hereinafter, the present application will be described in more detail.

An embodiment of the present application relates to a composition for enhancing immunity comprising at least one selected from the group consisting of a Lacticaseibacillus rhamnosus KBL352 strain deposited under Accession No. KCTC 15267BP, a culture of the strain, a lysate of the strain, and an extract of the strain. In Example of the present application, when a strain according to an embodiment of the present application was treated on macrophages, it activated the macrophages. Accordingly, the immunity enhancement may be activating macrophages, and the composition may be an immune stimulator.

Specifically, the composition may have one or more characteristics selected from the group consisting of the following (1) to (10):
(1) promotion of a phagocytic activity of macrophages, for example, the phagocytic activity of macrophages is 1.1-fold or higher, 1.2-fold or higher, 1.3-fold or higher, or 1.35-fold or higher, compared to an untreated control group,
(2) promotion of nitric oxide production by macrophages, for example, a nitric oxide production level of the macrophages is 1.1-fold or higher, 1.2-fold or higher, 1.3-fold or higher, 1.4-fold or higher, 1.5-fold or higher, 2-fold or higher, 2.5-fold or higher, 3-fold or higher, 3.5-fold or higher, 4-fold or higher, 5-fold or higher, 6-fold or higher, 7-fold or higher, 8-fold or higher, 10-fold or higher, 11-fold or higher, 12-fold or higher, 13-fold or higher, 14-fold or higher, or 15-fold or higher, compared to an untreated control group,
(3) promotion of cyclooxygenase-2 (COX-2) expression in macrophages,
(4) promotion of prostaglandin E2 (PGE₂) secretion by macrophages, for example, a secretion amount of PGE₂ of macrophages is 1.5-fold or higher, 1.8-fold or higher, 1.9-fold or higher, 2-fold or higher, 3-fold or higher, 4-fold or higher, 5-fold or higher, 8-fold or higher, 9-fold or higher, 10-fold or higher, 12-fold or higher, 14-fold or higher, 15-fold or higher, 20-fold or higher, or 25-fold or higher, compared to an untreated control group,
(5) promotion of cytokine secretion by macrophages, for example, a promotion of tumor necrosis factor-α (TNF-α) and/or interleukin-6 (IL-6) secretion,
(6) promotion of expression and phosphorylation of nuclear factor kappa B (NF-κB) and/or nuclear factor of kappa light polypeptide gene enhancer in B-cells inhibitor alpha (IκBα) of the macrophages,
(7) promotion of activation of mitogen-activated protein kinases (MAPKs) of the macrophages,
(8) prevention of body weight loss caused by immunosuppression,
(9) increase in a proportion of splenic natural killer (NK) cells, and
(10) increase in activity of the splenic NK cells.

Another embodiment of the present application relates to a food composition comprising the composition for enhancing immunity. The food may be a health functional food, and for example, may be in a form of meat products, bakery, chocolate, candies, jellies, snacks, confectionery, kimchi, fermented sauces, cheeses, dairy products, powders, beverages, or vitamin complexes.

Another embodiment of the present application relates to a quasi-drug composition comprising the composition for enhancing immunity. The quasi-drug composition may be in a form selected from the group consisting of toothpaste, mouthwash, oral spray, oral ointment, oral cleanser, oral deodorizer, bandage, and pest (mosquito, tick, and the like) repellent.

Another embodiment of the present application relates to a composition for preventing or treating immunosuppression, comprising at least one selected from the group consisting of the Lacticaseibacillus rhamnosus KBL352 strain deposited under Accession No. KCTC 15267BP, a culture of the strain, a lysate of the strain, and an extract of the strain.

The immunosuppression is caused by immune cells failing to defend against external pathogens or cancer cells due to immune deficiency, and may be, for example, immunodeficiency syndromes, and specifically, a secondary or an acquired immunodeficiency.

The immunosuppression may be one or more selected from the group consisting of herpes zoster, tuberculosis, meningitis, bronchiolitis, and bacterial or viral infections. In the present application, the term “strain” may refer to a live bacterium or heat-inactivated bacterium according to an embodiment of the present application.

In the present application, the term “culture of a strain” means a product obtained by culturing a strain according to an embodiment of the present application, and the culture may be a whole culture of the strain according to an embodiment of the present application, or a dilution, a concentrate, a dried product, a lyophilized product, a lysate, and/or a fraction thereof. The concentrate may be obtained by centrifuging or evaporating the culture, the dried product may be obtained by drying the culture using a dryer or the like, the lyophilized product may be obtained by lyophilizing the culture using a lyophilizer or the like, the lysates may be obtained by physically or ultrasonically treating the strain or the culture, and the fraction may be obtained by subjecting the culture, the lysate, and the like to a method such as centrifugation, chromatography, or the like. The culture may be in a solid phase (solid, for example, a dried product), a liquid phase (liquid), or a fluid phase, but is not limited thereto. In an example, the culture may mean the whole medium including the cultured strains, their metabolites, and/or extra nutrients, obtained by culturing the strain according to an embodiment of the present application for a certain period of time. In an example, the culture may be one in which the strain according to an embodiment of the present application is removed or not removed. In an example, the culture may mean the remaining components excluding the strain (bacteria) in the culture which is obtained by culturing the strain according to an embodiment of the present application in a medium. In an example, the culture may be a culture solution (or culture product) obtained by culturing the strain according to an embodiment of the present application in a medium from which the strains (bacteria) are removed. The culture solution (or culture product) from which the strains are removed may be a cell-free culture solution (or culture product) or a culture solution containing dead cells, and may be, for example, a filtrate (centrifuged supernatant) obtained by removing the strains through filtration or centrifugation, and/or a culture solution (or dried product of the culture solution) containing dead cells. Specifically, the culture may exhibit anti-inflammatory activity at a level equivalent to that exhibited by the strain according to an embodiment of the present application, or activity for preventing, improving or treating an inflammatory disease.

In the present application, the term “lysate of strain” may mean a product obtained by performing lysis a strain according to an embodiment of the present application by chemical or physical force. Specifically, the lysate may exhibit anti-inflammatory activity at a level equivalent to that exhibited by the strain according to an embodiment of the present application, or activity for preventing, improving, or treating inflammatory diseases.

The term “extract” in the present application may mean a product obtained by extracting a strain according to an embodiment of the present application, a culture of the strain, a lysate of the strain, or a mixture thereof, regardless of the extraction method, extraction solvent, extracted component, or form of the extract, and is a broad concept including all materials that can be obtained by processing or handling by another method after extraction. For example, the extract may be an extract of the strain according to an embodiment of the present application, an extract of the culture of the strain, or an extract of the lysate of the strain. Specifically, the extract may exhibit anti-inflammatory activity, or activity for preventing, improving, or treating inflammatory diseases, at a level equivalent to the activity exhibited by the strain according to an embodiment of the present application, a culture of the strain, or a lysate of the strain. The composition according to an embodiment of the present invention may include Lacticaseibacillus rhamnosus KBL352 strain deposited under Accession No. KCTC 15267BP at a concentration of 1.0×10³ to 1.0×10¹⁵ CFU, 1.0×10³ to 1.0×10¹⁴ CFU, 1.0×10³ to 1.0×10¹³ CFU, 1.0×10³ to 1.0×10¹² CFU, 1.0×10³ to 1.0×10¹¹ CFU, 1.0×10³ to 1.0×10¹⁰ CFU, 1.0×10⁵ to 1.0×10¹⁵ CFU, 1.0×10⁵ to 1.0×10^{14 CFU,} 1.0×10⁵ to 1.0×10¹³ CFU, 1.0×10⁵ to 1.0×10¹² CFU, 1.0×10⁵ to 1.0×10¹¹ CFU, 1.0×10⁵ to 1.0×10¹⁰ CFU, 1.0×10⁷ to 1.0×10¹⁵ CFU, 1.0×10⁷ to 1.0×10¹⁴ CFU, 1.0×10⁷ to 1.0×10¹³ CFU, 1.0×10⁷ to 1.0×10¹² CFU, 1.0×10⁷ to 1.0×10¹¹ CFU, 1.0×10⁷ to 1.0×10¹⁰ CFU, 1.0×10⁸ to 1.0×10¹⁵ CFU, 1.0×10⁸ to 1.0×10¹⁴ CFU, 1.0×10⁸ to 1.0×10¹³ CFU, 1.0×10⁸ to 1.0×10¹² CFU, 1.0×10⁸ to 1.0×10¹¹ CFU, or 1.0×10⁸ to 1.0×10¹⁰ CFU.

In the present application, the term ‘prevention’ means suppressing or delaying the onset of a disease, disorder, or condition. If the onset of a disease, disorder, or condition is suppressed or delayed for a predetermined period of time, prevention may be considered complete.

In the present application, the term ‘treatment’ means partially or completely alleviating, improving, alleviating, inhibiting, or delaying a specific disease, disorder, and/or condition or symptom, reducing the severity, or reducing the occurrence of one or more symptoms or characteristics.

The composition of the present application, for example, a pharmaceutical composition or a food composition, may additionally comprise one or more active ingredients exhibiting the same or similar function in addition to the above-mentioned active ingredients.

In addition, the composition according to the present application, for example, a pharmaceutical composition or a food composition, can be manufactured in a unit dosage form or manufactured by introducing into a multi-dose container by formulating using a pharmaceutically acceptable carrier according to a method that can be clearly performed by a person having ordinary skill in the art to which the present invention pertains. The term ‘carrier’ in the present application means a compound that facilitates the addition of a compound into cells or tissues and the term ‘pharmaceutically acceptable’ means a composition that is physiologically acceptable and does not typically cause an allergic reaction such as gastrointestinal disorder or dizziness or a similar reaction when administered to a human.

The pharmaceutically acceptable carriers mentioned above are those commonly used in the preparation of formulations and include lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methylcellulose, methylhydroxybenzoate, propylhydroxybenzoate, talc, magnesium stearate, and mineral oil, but are not limited to.

In addition, the composition according to the present application, for example, a pharmaceutical composition or a food composition, may additionally comprise additives such as a filler, an anticoagulant, a lubricant, a wetting agent, a flavoring agent, an emulsifier, a preservative, etc., in addition to the above components. In the present application, the content of the additives included in the composition is not particularly limited and may be appropriately adjusted within the content range used in conventional formulations.

In addition, the composition according to the present application, for example, a pharmaceutical composition or a food composition, may be formulated as an oral preparation. Non-limiting examples of the oral preparation include tablets, troches, lozenges, aqueous suspensions, oily suspensions, prepared powders, granules, emulsions, hard capsules, soft capsules, syrups, or elixirs. In order to formulate a pharmaceutical composition or a food composition according to the present application for oral administration, a binder such as lactose, saccharose, sorbitol, mannitol, starch, amylopectin, cellulose or gelatin; an excipient such as dicalcium phosphate; a disintegrant such as corn starch or sweet potato starch; magnesium stearate, calcium stearate, sodium stearyl fumarate, and the like; and a sweetener, a flavoring agent, a syrup, and the like can also be used. Furthermore, in the case of capsules, in addition to the above-mentioned substances, a liquid carrier such as fatty oil can be additionally used.

In the present application, the term ‘excipient’ means a substance other than a therapeutic agent, and means a carrier or medium used for delivering a therapeutic agent or added to a pharmaceutical composition. This improves handling and storage characteristics or allows and facilitates the formation of a unit dosage of the composition.

The composition according to the present application, for example, the pharmaceutical composition, may be formulated and used in various forms, such as oral formulations such as liquids, suspensions, powders, granules, tablets, capsules, pills, extracts, emulsions, syrups, aerosols, injections of sterile injectable solutions, and the like, according to a conventional method according to each intended use, and may be administered orally or through various routes including intravenous, intraperitoneal, subcutaneous, rectal, and topical administration. The term ‘oral administration’ in this application means a substance prepared so that an active substance can be digested, i.e., administered to the gastrointestinal tract for absorption.

The preferred dosage of a composition according to the present application, for example, a pharmaceutical composition or a food composition, may vary depending on the patient's condition and weight, age, sex, health condition, dietary constitution, nature of the preparation, degree of disease, administration time of the composition, administration method, administration period or interval, excretion rate, and drug form, and may be appropriately selected by a person skilled in the art.

The term ‘effective dosage’ in this application means the amount of a composition of an active ingredient sufficient to treat a specific symptom. This may vary depending on the formulation method of the pharmaceutical composition or food composition, the administration method, the administration time, and/or the administration route, and may vary depending on various factors including the type and degree of the response to be achieved by the administration of the pharmaceutical composition or food composition, the type, age, weight, general health condition, symptoms or degree of the disease, sex, diet, excretion, drugs used simultaneously or at the same time in the subject, other components of the composition, and similar factors well known in the medical field, and a person having ordinary skill in the art can easily determine and prescribe an effective dosage for the intended treatment.

The pharmaceutical composition or food composition according to the present application may be administered once a day or may be administered in several divided doses. The composition may be administered as an individual therapeutic agent or in combination with other therapeutic agents, and may be administered sequentially or simultaneously with conventional therapeutic agents. Taking all of the above factors into consideration, it may be administered in an amount that can obtain the maximum effect with the minimum amount without causing side effects.

For example, the composition according to the present application may be administered in a daily dosage of, but not limited to, 0.001 to 10,000 mg, 0.001 to 5,000 mg, 0.001 to 1,000 mg, 0.001 to 500 mg, 0.001 to 300 mg, 0.001 to 100 mg, 0.001 to 50 mg, 0.001 to 30 mg, 0.001 to 10 mg, 0.001 to 5 mg, 0.001 to 1 mg, 0.001 to 0.5 mg, 0.001 to 0.1 mg, 0.001 to 0.05 mg, 0.001 to 0.01 mg, 0.01 to 10,000 mg, 0.01 to 5,000 mg, 0.01 to 1,000 mg, 0.01 to 500 mg, 0.01 to 300 mg, 0.01 to 100 mg, 0.01 to 50 mg, 0.01 to 30 mg, 0.01 to 10 mg, 0.01 to 5 mg, 0.01 to 1 mg, 0.01 to 0.5 mg, 0.01 to 0.1 mg, 0.01 to 0.05 mg, 0.1 to 10,000 mg, 0.1 to 5,000 mg, 0.1 to 1,000 mg, 0.1 to 500 mg, 0.1 to 300 mg, 0.1 to 200 mg, 0.1 to 100 mg, 0.1 to 50 mg, 0.1 to 30 mg, 0.1 to 10 mg, 0.1 to 5 mg, 0.1 to 1 mg, 0.1 to 0.5 mg, 1 to 10,000 mg, 1 to 5,000 mg, 1 to 1,000 mg, 1 to 500 mg, 1 to 300 mg, 1 to 200 mg, 1 to 100 mg, 1 to 50 mg, 1 to 10 mg, 1 to 5 mg, 10 to 10,000 mg, 10 to 5,000 mg, 10 to 1,000 mg, 10 to 500 mg, 10 to 300 mg, 10 to 200 mg, 10 to 100 mg, 10 to 50 mg, 10 to 40 mg, 10 to 30 mg, 10 to 20 mg, 100 to 10,000 mg, 100 to 5,000 mg, 100 to 1,000 mg, 100 to 500 mg, 100 to 300 mg, or 100 to 200 mg per 1 kg of body weight. For example, the daily dosage of the composition according to the present application may be 0.001 to 10 g/day, 0.001 to 5 g/day, 0.01 to 10 g/day, or 0.01 to 5 g/day based on oral administration to an adult patient. In addition, the total daily dosage may be divided and administered continuously or discontinuously as needed.

Another embodiment of the present application relates to a method for enhancing immunity, comprising a step of administering to a subject at least one selected from the group consisting of Lacticaseibacillus rhamnosus KBL352 strain deposited under Accession No. KCTC 15267BP, a culture of the strain, a lysate of the strain, and an extract of the strain.

### Advantageous Effects

Lacticaseibacillus rhamnosus KBL352 strain deposited under Accession No. KCTC 15267BP according to an embodiment of the present application can enhance a phagocytic activity of macrophages and increase activity of the macrophages to enhance immunity, so that it can be utilized for immunity enhancement, can provide effects of preventing and treating immunosuppression, alleviating side effects caused by taking immunosuppressants, antivirus, and preventing infectious diseases, and can be usefully utilized as a probiotic material.

### DESCRIPTION OF DRAWINGS

FIG. 1A and FIG. 1B are drawings confirming that a phagocytic activity of macrophages is enhanced by a strain according to an embodiment of the present application.

FIG. 2A is a drawing confirming that the expression of inducible nitric oxide synthase (iNOS) in macrophages is increased by a strain according to an embodiment of the present application.

FIG. 2B is a drawing confirming that the NO secretion in macrophages is increased by a strain according to an embodiment of the present application.

FIG. 3A is a drawing confirming that the expression of COX-2 in macrophages is increased by a strain according to an embodiment of the present application.

FIG. 3B is a drawing confirming that the secretion of PGE₂ in macrophages is increased by a strain according to an embodiment of the present application.

FIG. 4A and FIG. 4B are drawings confirming that the secretion of cytokines in macrophages is increased by a strain according to an embodiment of the present application.

FIG. 5 is a drawing confirming that expression of NF-κB and expression and phosphorylation of IκBα are increased in macrophages by a strain according to an embodiment of the present application.

FIG. 6 is a drawing confirming that the phosphorylation of c-Jun N-terminal kinases (JNK), extracellular signal-regulated kinase (ERK), and p38 proteins are increased in macrophages by a strain according to an embodiment of the present application.

FIG. 7A is a drawing confirming an effect of weight loss prevention of a strain according to an embodiment of the present application in an immunosuppressed animal model.

FIG. 7B is a drawing confirming an effect of increase in splenic NK cells due to the strain according to an embodiment of the present application in an immunosuppressed animal model.

FIG. 7C is a drawing confirming the increase in activity of the splenic NK cells due to the strain according to an embodiment of the present application in an immunosuppressed animal model.

### MODES FOR INVENTION

Hereinafter, the present application will be described in more detail through the following Examples. However, these Examples are only intended to be illustrative of the present application, and the scope of the present application is not to be limited by these Examples.

### Example 1. Identification of a Lacticaseibacillus rhamnosus KBL352 Strain

A fecal sample from 3-month-old infant was provided through Seoul Samsung Medical Center, and the sample was inoculated onto a selective medium (TOS-propionate agar medium; 43314 Transgalactosylated oligiosaccharide agar medium, Fluka), cultured in an anaerobic environment at 37° C. for 48 hours, and pure cultured using the colony-picking method.

Then, strains were cultured in the anaerobic environment in a CO₂ incubator at 37° C. for one day, respectively, and centrifugation was performed at 13,000 rpm at 4° C. to obtain the bacterial pellet only. Then, 0.2 μL of the bacterial pellet was added to 25 μL of lysis buffer (containing pH 10; 2.5 M NaCl, 100 mM EDTA, 10 mM Trizma base, and 1% Triton X-100) and heat treated at 95° C. for 10 minutes to obtain the bacterial genome. Thereafter, a V4 region of 16S rRNA was amplified by a polymerase chain reaction using a G-Taq PCR kit to obtain a PCR product. Base sequences of primers used are shown in Table 1. In the base sequences of the primers below, “M” is selected from A or C, and “Y” is selected from C or T.

TABLE 1

| | Sequence | SEQ ID |
|---|---|---|
| Classification | (5′ -> 3′) | NO. |
| 27F forward | AGAGTTTGATCMTG | 1 |
| primer | GCTCAG | |
| 1492R | TACGGYTACCTTGT | 2 |
| reverse | TACGACTT | |
| primer | AGCTCGTGTCGTGA | 3 |
| 16S rRNA | GATGTTGGGTTAAG | |
| | TCCCGCAACGAGC | |
| | GCAACCCTTATGA | |
| | CTAGTTGCCAGCA | |
| | TTTAGTTGGGCAC | |
| | TCTAGTAAGACTG | |
| | CCGGTGACAAACC | |
| | GGAGGAAGGTGGG | |
| | GATGACGTCAAAT | |
| | CATCATGCCCCTT | |
| | ATGACCTGGGCTA | |
| | CACACGTGCTACA | |
| | ATGGATGGTACAA | |
| | CGAGTTGCGAGAC | |
| | CGCGAGGTCAAGC | |
| | TAATCTCTTAAAG | |
| | CCATTCTCAGTTC | |
| | GGACTGTAGGCTG | |
| | CAACTCGCCTACA | |
| | CGAAGTCGGAATC | |
| | GCTAGTAATCGCG | |
| | GATCAGCACGCCG | |
| | CGGTGAATACGTT | |
| | CCCGGGCCTTGTA | |
| | CACACCGCCCGTC | |
| | ACACCATGAGAGT | |
| | TTGTAACACCCGA | |
| | AGCCGGTGGCGTA | |
| | ACCCTTTTAGGGA | |
| | GCGAGCCGTCTAA | |
| | GGTGGGACAAATG | |
| | ATTAGGGTGAAGT | |
| | CGTAAC | |

Then, the PCR product was purified using an Ultra Clean PCR clean-up Kit (Mobio Laboratories Inc.), and gene sequencing was requested to Macrogen to identify the strain. A 16S rRNA sequence of the strain is shown in SEQ ID NO. 3.

As a result, the identified bacteria was confirmed to be a novel Lacticaseibacillus sp. strain, and the strain was named the Lacticaseibacillus rhamnosus KBL352 strain. The strain was deposited to Korean Collection for Type Culture of Korea Research Institute of Bioscience and Biotechnology that is an international depository authority under the Budapest Treaty, and was assigned an accession number of KCTC 15267BP.

### Example 2: Effect of Improving a Phagocytic Activity of Macrophages

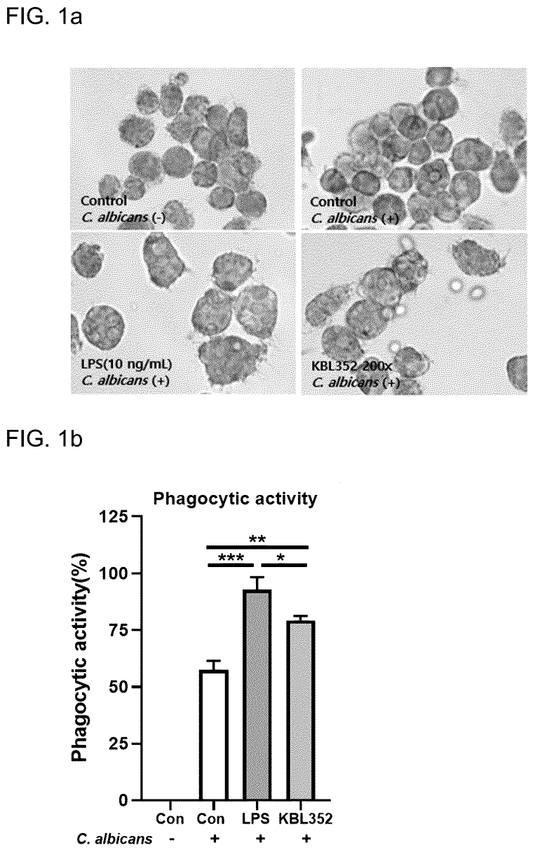

A phagocytic activity of macrophages was analyzed using Candida albicans MYA-4788 (hereinafter C. albicans). A mouse macrophage cell line RAW264.7 was treated with live bacteria of Lacticaseibacillus rhamnosus KBL352 at a 200-fold ratio and co-cultured for 24 hours, and then, RAW264.7 cells were washed two times with 1×PBS. As a control group, lipopolysaccharide (LPS) was treated at a concentration of 10 ng/ml. Subsequently, RAW264.7 cells were treated with the C. albicans in a yeast state at a 10-fold level and cultured at 37° C. for 1 hour. The RAW264.7 cells that have been co-cultured with the C. albicans were washed two times with the 1×PBS, fixed by treating with 2.5% glutaraldehyde, and stained by treating with 0.5% methyleneblue dye. The stained cells were observed under a microscope. When one or more of the C. albicans were seen in the macrophages, it was considered that a phagocytosis had occurred. Results of the microscope observation are shown in FIG. 1A, and the phagocytic activity of the macrophages was calculated using the following Equation 1 and shown in FIG. 1B and Table 2:

$$Phagocytic activity ( \% ) = ( Number of macrophages in which a phagocytosis has occurred ) / ( number of observed macrophages ) \times 100 \qquad [ Equation 1 ]$$

TABLE 2

| Classification | Phagocytic activity(%) |
|---|---|
| Untreated group | 57.4 |
| LPS | 92.8 |
| KBL352 | 79.3 |

As shown in FIGS. 1A, 1B, and Table 2, a significant increase in the phagocytic ability of the macrophages was confirmed when treated with the live bacteria of the Lacticaseibacillus rhamnosus KBL352.

### Example 3. Effect of Increasing iNOS Expression and NO Production of Macrophages

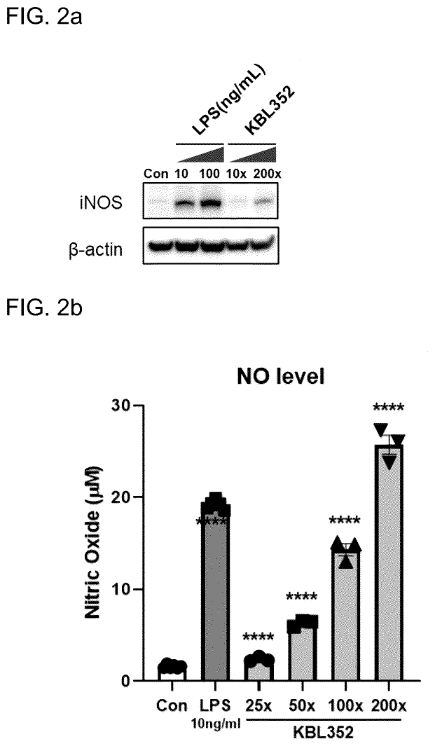

Mouse macrophage cell line RAW264.7 was treated with live bacteria of Lacticaseibacillus rhamnosus KBL352 at a concentration gradient of up to 200-fold compared to that of RAW264.7 cells. The expression of iNOS, a protein involved in NO production and a NO secreted by macrophages were measured. As a control group, LPS was treated at a concentration of 10 ng/ml or 100 ng/ml, and an expression level of β-actin that is a housekeeping protein, was observed together to confirm that a protein used in FIG. 2A was constant in all samples. The expression of iNOS is shown in FIG. 2A and the amount of NO secreted by macrophages is shown in FIG. 2B and Table 3.

TABLE 3

| Classification | NO concentration(μM) |
|---|---|
| Untreated group | 1.7 |
| LPS | 18.85 |
| KBL352 25x | 2.38 |
| KBL352 50x | 6.31 |
| KBL352 100x | 14.31 |
| KBL352 200x | 25.71 |

As shown in FIG. 2A, FIG. 2B, and Table 3, the iNOS expression and the NO secretion amount were increased depending on a strain treatment level, indicating that a strain according to an example of the present application is an immune stimulant that activates immune cells.

### Example 4. Confirmation of Increase in COX-2 Expression and PGE₂ Secretion in Macrophages

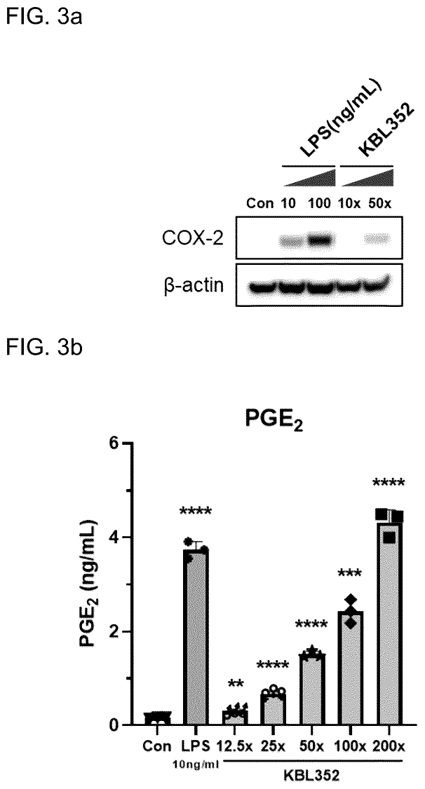

Mouse macrophage cell line RAW264.7 was treated with live bacteria of Lacticaseibacillus rhamnosus KBL352 at a concentration gradient of up to 200-fold compared to that of RAW264.7 cells. The expression of COX-2, a protein involved in PGE₂ production, and a PGE₂ secretion of macrophages were measured. As a control group, LPS was treated at a concentration of 10 ng/ml or 100 ng/ml. The expression level of β-actin, a housekeeping protein, was observed together to confirm that a protein used in FIG. 2A was constant in all samples. The COX-2 expression is shown in FIG. 3A and the PGE₂ secretion of macrophages is shown in FIG. 3B and Table 4.

TABLE 4

| Classification | PGE₂ (ng/ml) |
|---|---|
| Untreated group | 0.16 |
| LPS | 3.73 |
| KBL352 12.5x | 0.31 |
| KBL352 25x | 0.67 |
| KBL352 50x | 1.51 |
| KBL352 100x | 2.43 |
| KBL352 200x | 4.31 |

As shown in FIG. 3A, FIG. 3B, and Table 4, the COX-2 expression and the PGE₂ secretion were increased depending on a strain treatment level according to an example of the present application, indicating that a strain according to an example of the present application is an immune stimulant that activates the immune cells.

### Example 5. Effect of Increasing Cytokine Production in Macrophages

Mouse macrophage cell line RAW264.7 was treated with live bacteria of Lacticaseibacillus rhamnosus KBL352 at a concentration gradient of up to 200-fold compared to that of RAW264.7 cells. The production TNF-α and IL-6 of macrophages were measured. As a positive control group, LPS was treated at a concentration of 10 ng/mL.

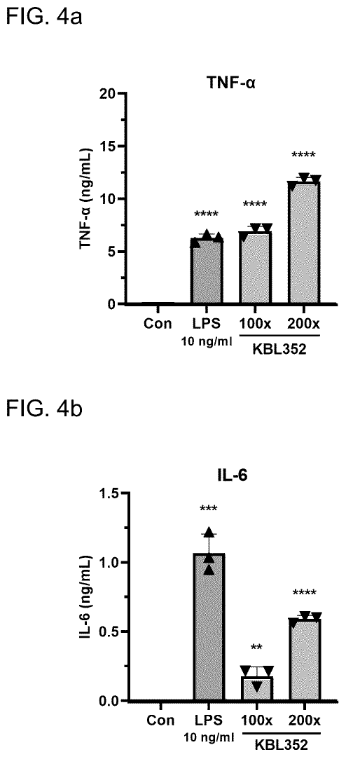

As shown in FIG. 4A, FIG. 4B, and Table 5, TNF-α and IL-6 in the macrophages increased depending on a strain treatment level according to an example of the present application, indicating that a strain according to an example of the present application has an immune function enhancement effect.

TABLE 5

| Classification | TNF-α (ng/mL) | IL-6 (ng/mL) |
|---|---|---|
| Untreated group | 0 | 0 |
| LPS | 6.268 | 1.066 |
| KBL352 100x | 6.918 | 0.177 |
| KBL352 200x | 11.643 | 0.591 |

### Example 6. Increasing Effect of Expression and Phosphorylation of NF-κB and IκBα in Macrophages

Mouse macrophage cell line RAW264.7 was treated live bacteria of Lacticaseibacillus rhamnosus KBL352 at a concentration gradient of 10 to 50-fold. The expression and phosphorylation of NF-κB and IκBα proteins were confirmed. As a control, LPS was treated at a concentration of 10 ng/ml or 100 ng/ml. The expression level of β-actin, a housekeeping protein, was observed together to confirm that a protein used was constant in all samples.

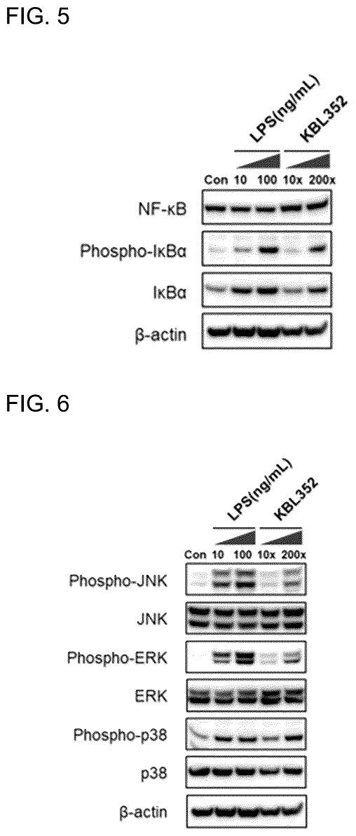

As shown in FIG. 5, the NF-κB expression and the IκBα expression and phosphorylation thereof were increased by a strain treatment according to an example of the present application, and it was confirmed that immune ability was activated.

### Example 7. Effect of MAPKs Activation in Macrophages

Mouse macrophage cell line RAW264.7 was treated with live bacteria of Lacticaseibacillus rhamnosus KBL352 at a concentration gradient of 10 to 200-fold compared to that of RAW264.7 cells. The expression and phosphorylation levels of MAPKs (p38, ERK, JNK), which are important signaling pathways for activation of the immune cells by promoting NO production and the secretion of cytokines such as TNF-α and IL-6 in immune cells, were confirmed. As a control group, LPS was treated at a concentration of 10 ng/ml or 100 ng/ml. An expression level of β-actin, a housekeeping protein, was observed together to confirm that a protein used was constant in all samples.

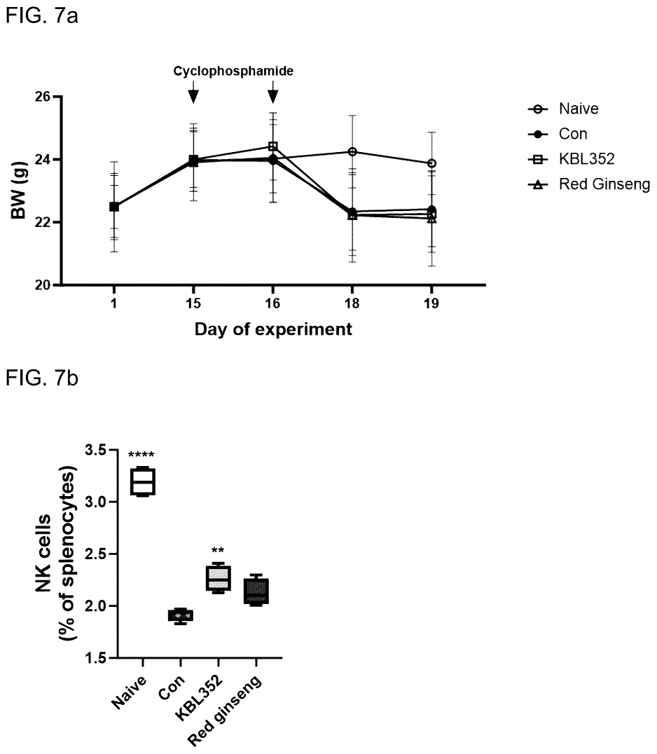

As shown in FIG. 6, phosphorylation levels of JNK, ERK, and p38 proteins were increased by a strain treatment according to an example of the present application, and it was confirmed that immune ability was activated.

### Example 8. Effect of Immunity Enhancement

### (1) Preparation of an Immunosuppressed Animal Model

Water, Lacticaseibacillus rhamnosus KBL352 strain, or a red ginseng concentrate were orally administered into five-week-old male Balb/c mice once a day by group according to Table 6 until the end of the experiment. On days 15 and 16 of the experiment, cyclophosphamide was administered into the intraperitoneal of mice except for the naïve group at a concentration of 150 mg/kg, respectively, to induce immunosuppression.

TABLE 6

| | Administered | |
|---|---|---|
| Group | amount | Concentration |
| Naïve | Water 200 μL | — |
| Control | Water 200 μL | — |
| KBL352 | 200 μL | Diluted in water to 10\hat{ }9 CFU/mL |
| Red | 200 μL (200 | 12.7 mg/g of ginsenosides including Rg1, |
| ginseng | mg/kg) | Rb1, and Rg3 was contained |

### (2) Effect of Weight Loss Prevention

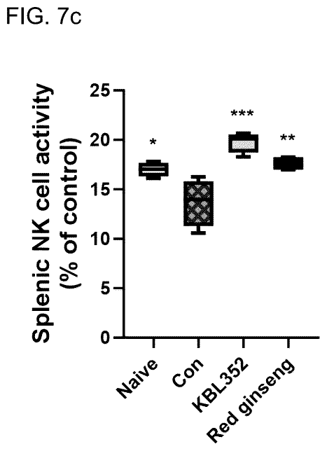

Weights of the mice were measured before the oral administration once a day from day 1 of the experiment to the end and are shown in FIG. 7A. As shown in FIG. 7A, Lacticaseibacillus rhamnosus KBL352 strain according to an example of the present application showed an effect of preventing weight loss due to immunity suppression.

### (3) Effect of Increasing a Proportion of Splenic NK Cells

Spleens of each group of the mice were obtained after the end of the experiment and a number of splenic NK cells was confirmed through a flow cytometer and is shown in FIG. 7B. As shown in FIG. 7B, it was confirmed that the splenic NK cells were significantly increased in the group administered with the Lacticaseibacillus rhamnosus KBL352 strain according to an example of the present application, and in particular, it showed an effect equal to or greater than that of a red ginseng. Accordingly, the Lacticaseibacillus rhamnosus KBL352 strain according to an example of the present application has a remarkably excellent immune enhancement effect that induces rapid recovery from immunity suppression. Compared to the control group in FIG. 7B, * indicates p<0.05, ** indicates p<0.01, *** indicates p<0.001, and **** indicates p<0.0001.

### (4) Effect of Increasing Activity of Splenic NK Cells

The spleens of each group of the mice were obtained after the end of the experiment. A proportion of Yac-1 cells, target cells of the NK cells, killed by the NK cells was confirmed through the flow cytometer and is shown in FIG. 7C. As shown in FIG. 7C, the proportion of the splenic NK cells significantly increased in the group administered with the Lacticaseibacillus rhamnosus KBL352 strain according to an example of the present application. It showed an effect equal to or even greater than that of the red ginseng. Accordingly, the Lacticaseibacillus rhamnosus KBL352 strain according to an example of the present application has a remarkably excellent immune enhancement effect that induces rapid recovery from immunity suppression. Compared to the control group in FIG. 7C, * indicates p<0.05, ** indicates p<0.01, *** indicates p<0.001, and **** indicates p<0.0001.

### [Accession Number]

Depositary Authority: Korean Collection for Type Cultures (KCTC), Korea Research Institute of Bioscience and Biotechnology (KRIBB)
Accession Number: KCTC15267BP
Date of Deposit: Jan. 4, 2023

1.-8. (canceled)

9. A method for enhancing immunity, the method comprising a step of administering to a subject a composition comprising a Lacticaseibacillus rhamnosus KBL352 strain deposited under Accession No. KCTC 15267BP, a culture of the strain, a lysate of the strain, or an extract thereof.

10. The method according to claim 9, wherein the enhancing immunity promotes phagocytic activity of macrophages and/or promotes secretion of nitric oxide of the macrophages.

11. The method according to claim 9, wherein the composition is an immune-stimulator.

12. The method according to claim 9, wherein the composition has at least one of the following characteristics:
(1) promotion of a phagocytic activity of macrophages,
(2) promotion of NO secretion by macrophages,
(3) promotion of COX-2 expression in macrophages,
(4) promotion of PGE₂ secretion by macrophages,
(5) promotion of cytokine secretion by macrophages,
(6) promotion of expression and phosphorylation of NF-κB and/or IκBα in macrophages,
(7) promotion of activation of MAPKs in macrophages,
(8) prevention of body weight loss caused by immunosuppression,
(9) increase in a proportion of splenic NK cells, and
(10) increase in activity of the splenic NK cells.

13. A Lacticaseibacillus rhamnosus KBL352 strain deposited under Accession No. KCTC 15267BP.

14. The strain according to claim 13, wherein the strain comprises a 16S rRNA sequence set forth in SEQ ID NO. 3.

15. A composition in the form of tablets, pills, troches, lozenges, aerosols, suspensions, powders, granules, emulsions, capsules, syrups, or elixirs, comprising the strain according to claim 13, a culture of the strain, a lysate of the strain, or an extract thereof.

16. A food comprising the strain according to claim 13, a culture of the strain, a lysate of the strain, or an extract thereof.

17. A quasi-drug comprising the strain according to claim 13, a culture of the strain, a lysate of the strain, or an extract thereof.


SEQUENCE LISTING (3 sequences)

SEQ ID NO: 1 - DNA, 20 bp, synthetic construct
  misc_feature 1..20: note = 27F forward primer
  source 1..20: mol_type = other DNA
agagtttgatcmtggctcag

SEQ ID NO: 2 - DNA, 22 bp, synthetic construct
  misc_feature 1..22: note = 1492R reverse primer
  source 1..22: mol_type = other DNA
tacggytaccttgttacgactt

SEQ ID NO: 3 - DNA, 1458 bp, Lacticaseibacillus rhamnosus
  source 1..1458: mol_type = genomic DNA
tctgattattgaaaggtgcttgcttcttgatttaattttgaacgagtggcggacgggtgagtaacacgtgggtaacctgcccttaagtgggggataacatttggaaacagatgctaataccgcataaatccaagaaccgcatggttcttggctgaaagatggcgtaagctatcgcttttggatggacccgcggcgtattagctagttggtgaggtaacggctcaccaaggcaatgatacgtagccgaactgagaggttgatcggccacattgggactgagacacggcccaaactcctacgggaggcagcagtagggaatcttccacaatggacgcaagtctgatggagcaacgccgcgtgagtgaagaaggctttcgggtcgtaaaactctgttgttggagaagaatggtcggcagagtaactgttgtcggcgtgacggtatccaaccagaaagccacggctaactacgtgccagcagccgcggtaatacgtaggtggcaagcgttatccggatttattgggcgtaaagcgagcgcaggcggttttttaagtctgatgtgaaagccctcggcttaaccgaggaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctggtagtccatgccgtaaacgatgaatgctaggtgttggagggtttccgcccttcagtgccgcagctaacgcattaagcattccgcctggggagtacgaccgcaaggttgaaactcaaaggaattgacgggggcccgcacaagcggtggagcatgtggtttaattcgaagcaacgcgaagaaccttaccaggtcttgacatcttttgatcacctgagagatcaggtttccccttcgggggcaaaatgacaggtggtgcatggttgtcgtcagctcgtgtcgtgagatgttgggttaagtcccgcaacgagcgcaacccttatgactagttgccagcatttagttgggcactctagtaagactgccggtgacaaaccggaggaaggtggggatgacgtcaaatcatcatgccccttatgacctgggctacacacgtgctacaatggatggtacaacgagttgcgagaccgcgaggtcaagctaatctcttaaagccattctcagttcggactgtaggctgcaactcgcctacacgaagtcggaatcgctagtaatcgcggatcagcacgccgcggtgaatacgttcccgggccttgtacacaccgcccgtcacaccatgagagtttgtaacacccgaagccggtggcgtaacccttttagggagcgagccgtctaaggtgggacaaatgattagggtgaagtcgtaac